(19) United States
(12) Patent Application Publication
(10) Pub. No.: US 2024/0327315 A1
(43) Pub. Date: Oct. 3, 2024

(54) Diels-Alder coupling for cycloalkane production for sustainable aviation fuel

(71) Applicants: Alliance for Sustainable Energy, LLC, Golden, CO (US); Massachusetts Institute of Technology, Cambridge, MA (US)

(72) Inventors: Gregg Tyler BECKHAM, Golden, CO (US); Julia Bryant CURLEY, Denver, CO (US); Yuriy ROMÁN, Cambridge, MA (US); David Gregory BRANDNER, Golden, CO (US); Ciaran William LAHIVE, Golden, CO (US)

(21) Appl. No.: 18/623,523

(22) Filed: Apr. 1, 2024

Related U.S. Application Data
(60) Provisional application No. 63/493,425, filed on Mar. 31, 2023.

(51) Int. Cl. C07C 2/52; C07C 1/20; C07C 5/03; C10L 1/06

(52) U.S. Cl. CPC C07C 2/52; C07C 1/20; C07C 5/03; C10L 1/06

(57) ABSTRACT

Described herein are methods for the generation of cyclic alkanes, useful as sustainable aviation fuel, from sustainable biomass sources. The described methods utilize Diels-Alder reaction followed by hydrogenation to generate the desired compounds from lignocellulosic biomass.

### CROSS-REFERENCE TO RELATED APPLICATIONS

This application claims priority from U.S. Provisional Patent Application No. 63/493,425 filed on Mar. 31, 2023, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

A significant contribution to greenhouse emissions is the burning of fossil fuels in the aviation industry. Accordingly, the generation of economically viable sustainable aviation fuel (SAF) from non-petroleum based sources, including biomass, is essential to reduce carbon emissions. A key component of sustainable aviation fuel is jet-range cycloalkanes. Currently routes to SAF at scale today mostly focus on bio-based linear and isoalkanes. For example, current ethanol upgrading undergoes dehydration, oligomerization, and isomerization to linear and branched alkanes, but there is a need for a substantial amount of cycloalkanes in SAF, which technologies at scale today cannot meet. This technology would allow for a single-step route to produce cycloalkenes, which could then be hydrogenated to produce cycloalkanes in the jet fuel range. It can be seen from the foregoing that there remains a need in the art for generation of sustainable aviation fuel in the form of cycloalkanes from biomass, specifically lignocellulosic biomass.

### SUMMARY

Described herein are methods for the generation of cyclic alkanes, useful as sustainable aviation fuel, from sustainable biomass sources. The described methods utilize Diels-Alder reaction followed by hydrogenation to generate the desired compounds from lignocellulosic biomass.

Diels-Alder coupling reactions occur between dienes and alkenes to produce cyclic compounds. This chemistry has been known for many decades in synthetic organic chemistry. Here, we are proposing to use Diels-Alder coupling with bio-based dienes and bio-based alkenes to produce cycloalkenes, which can be hydrogenated, to produce much-needed cycloalkanes for sustainable aviation fuel (SAF). There are multiple likely routes for this type of chemistry to be viable.

Examples of chemical routes for SAF include: Coupling of a bio-based diene to a bio-based medium or long-chain olefinic fatty acid. Examples include ethanol-derived butadiene, furans, cyclopentadienone, or other dienes coupled to olefinic acids derived from dehydrated 3-hydroxyacids or from cultivations of oleaginous microbes that produce olefinic fatty acids. As well as, Coupling of a bio-based diene to mixtures of bio-based olefins. Examples include mixtures of butenes derived from ethanol (or butenes, hexenes, octenes from ethanol) coupled to any of the bio-based dienes listed herein.

In an aspect, provided is a method comprising: a) providing lignocellulosic biomass; b) converting a portion of the lignocellulosic biomass into one or more dienes comprising butadiene, isoprene, one or more cyclic dienes, or a combination thereof; c) converting a portion of the lignocellulosic biomass into one or more dienophiles comprising olefins, unsaturated fatty acids or a combination thereof; d) reacting the one or more dienes and the one or more dienophiles in the presence of a catalyst, thereby generating one or more cyclic olefins; and e) hydrogenating the one or more cyclic olefins, thereby generating one or more cyclic alkanes.

The step of reacting to form the one or more dienes may be performed by a catalyzed Diels-Alder reaction. Similarly, the step of reacting to form the one or more dienophiles may be performed by a catalyzed Diels-Alder reaction. The described Diels-Alder reactions may be heterogeneous or homogeneous.

The step may be performed in multiple steps or as a single step followed by a separation. The step of hydrogenating may performed via a heterogeneous or homogeneous hydrogenation reaction. The step of converting the lignocellulosic biomass into one or more dienes or one or more dienophiles may be performed in the presence of a Zn—Y catalyst.

The one or more dienes may comprise butadiene, isoprene, may be defined by the formulas:

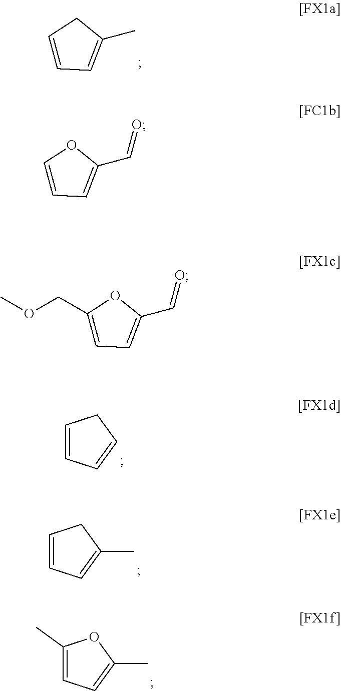

or a combination thereof.

The one or more dienophiles may be olefins or unsaturated fatty acids. For example, the one or more dienophiles may be defined by the formulas:

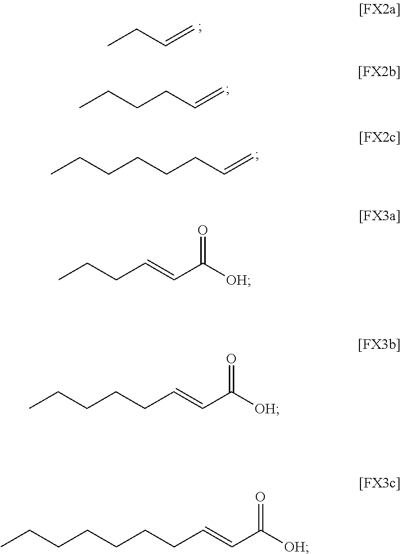

or a combination thereof.

The one or more dienophiles may comprise one or more molecules defined by the formulas:

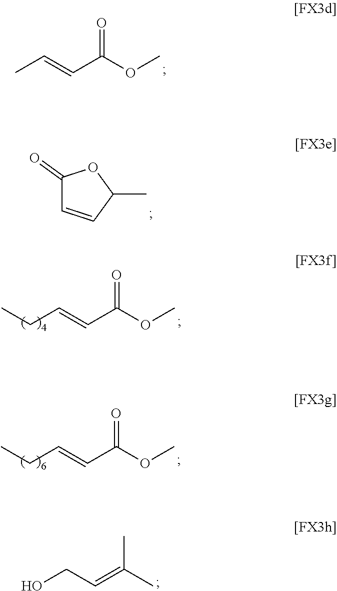

or a combination thereof.

The one or more cycloalkanes may comprise sustainable aviation fuel (SAF) or cycloalkanes in the SAF range. For example, the cycloalkanes may be described by the formula:

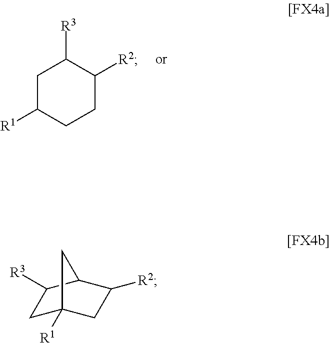

wherein each of R¹, R² and R³ are independently selected from an H atom or a C1-C8 alkane. The one or more cycloalkanes may also be described by the formula:

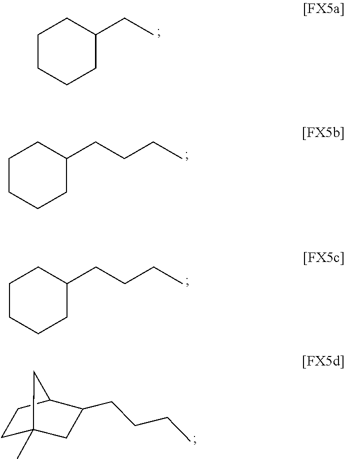

or a combination thereof.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 provides an example synthesis scheme for the generation of cycloalkanes. The follow reaction steps are described: 1) C-CBP cotreatment consolidated bioprocessing, 2) Zn—Y/Beta catalyzed conversion, 3) Zn—Y/Beta and single-atom alloy composite catalyzed conversion; 4) Homogeneous or heterogeneous catalyzed Diels-Alder reaction; and 5) Homogeneous or heterogeneous catalyzed olefin hydrogenation.

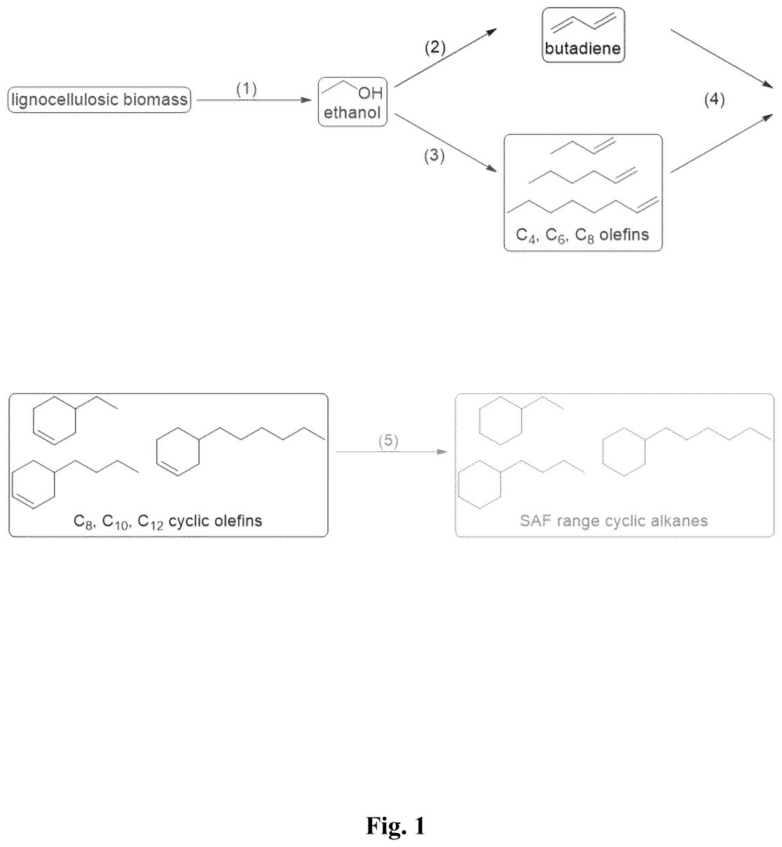

FIG. 2 provides an example synthesis scheme for the generation of cycloalkanes. Similar reaction steps provided in FIG. 1 may be implemented in FIG. 2.

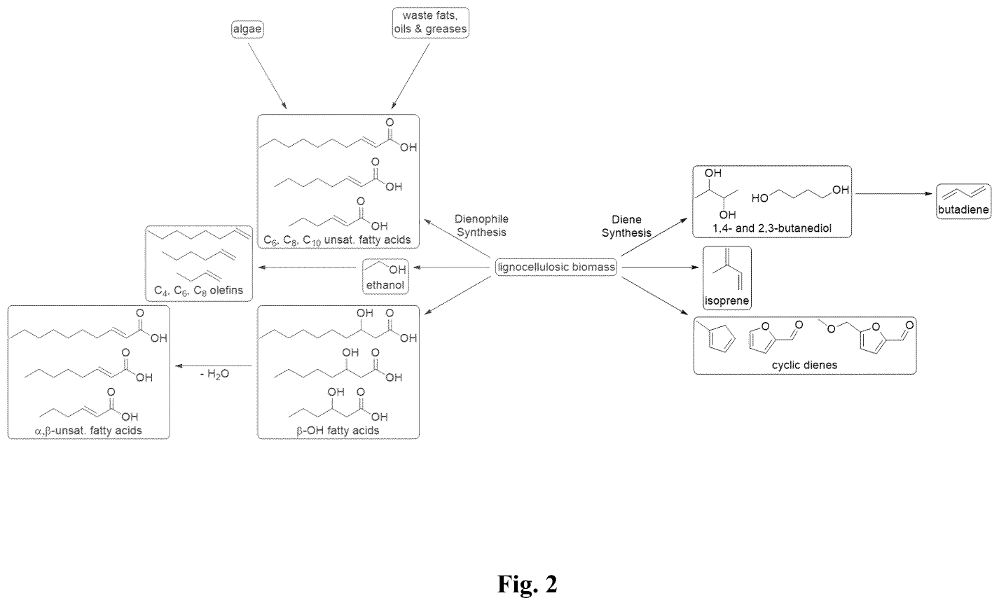

FIG. 3 provides product formation from the preliminary scheme steps provided in FIG. 2.

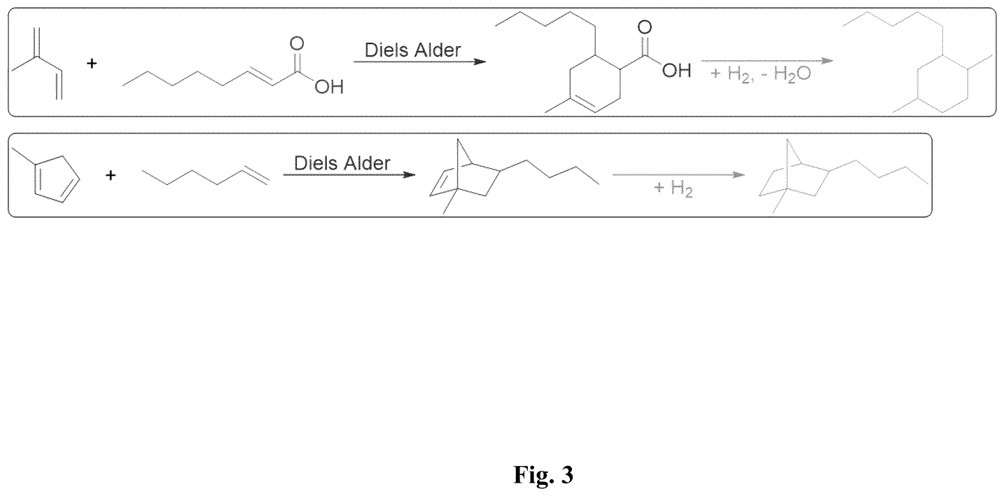

FIG. 4 provides an example reaction for generating cycloalkanes (top) and base nuclear magnetic resonance (NMR) data for the 1-octene reactant (bottom).

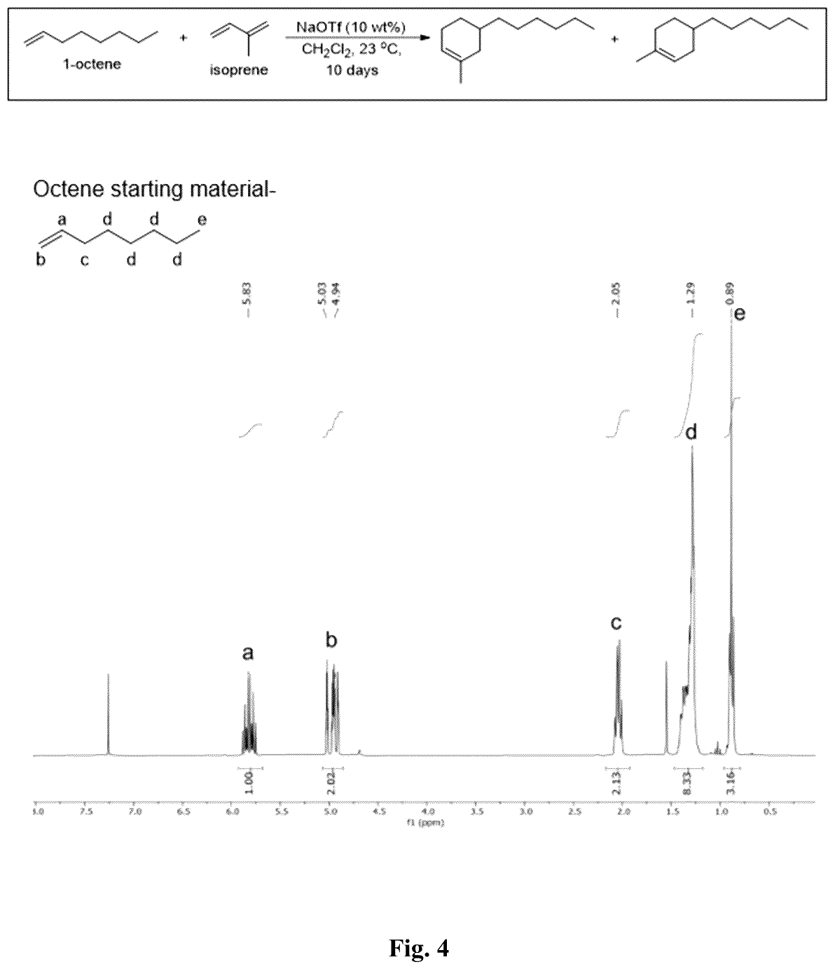

FIG. 5 provides NMR data for the reaction described in FIG. 4 after 8 days. The additional peaks with respect to FIG. 4 illustrate product formation.

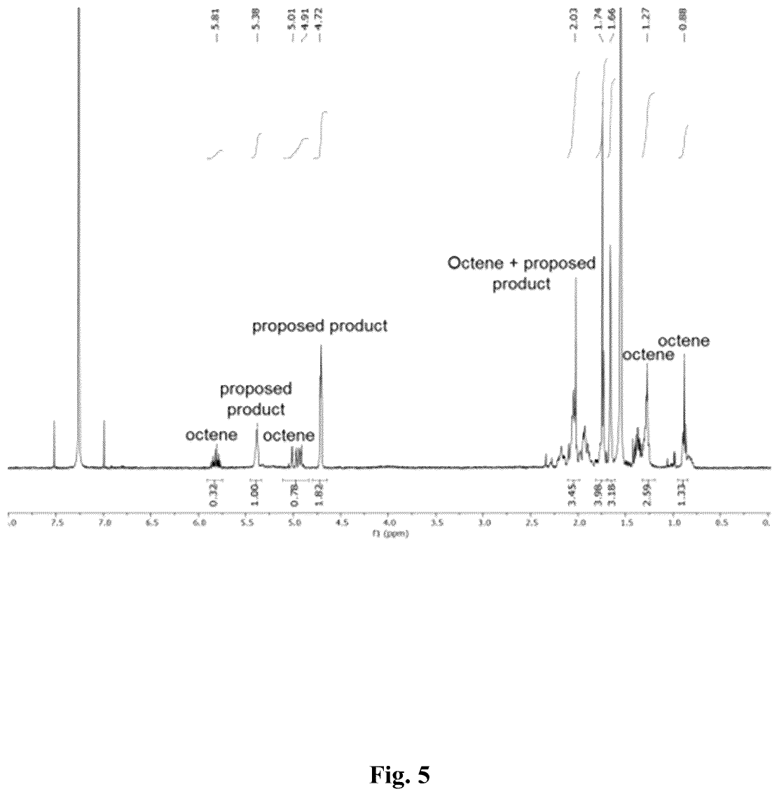

FIG. 6 provides NMR data for the reaction described in FIG. 4 an additional 2 days from the data in FIG. 5, after removing the reactant solvent. FIG. 6 illustrates that no 1-octene reactant remains.

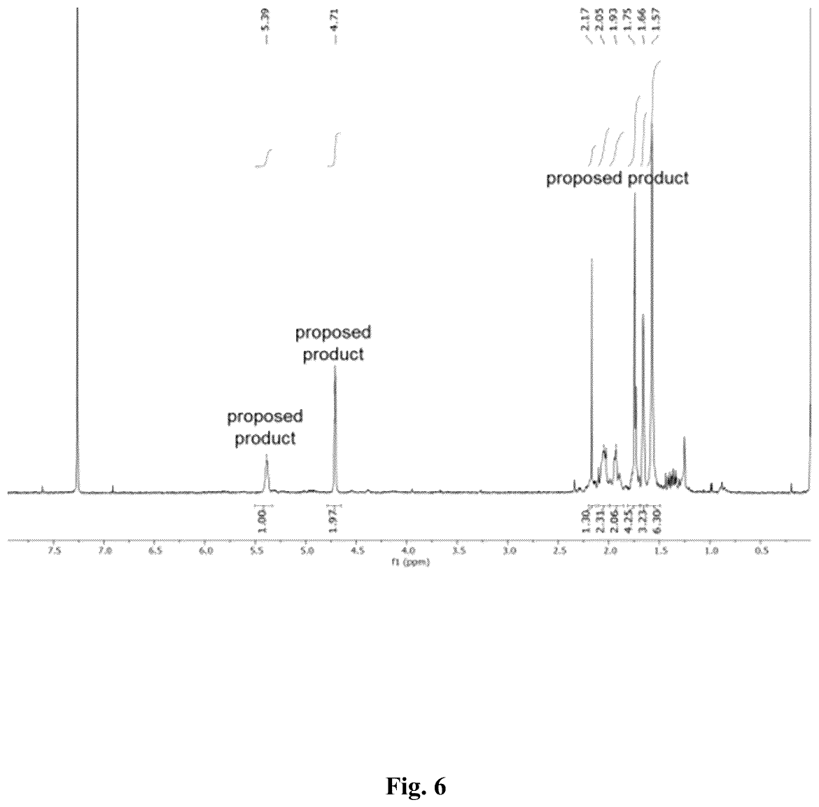

FIG. 7 provides an example reaction for generating cycloalkanes (top) and base nuclear magnetic resonance (NMR) data for the ethyl trans-2-decenoate (Et2d) reactant (bottom).

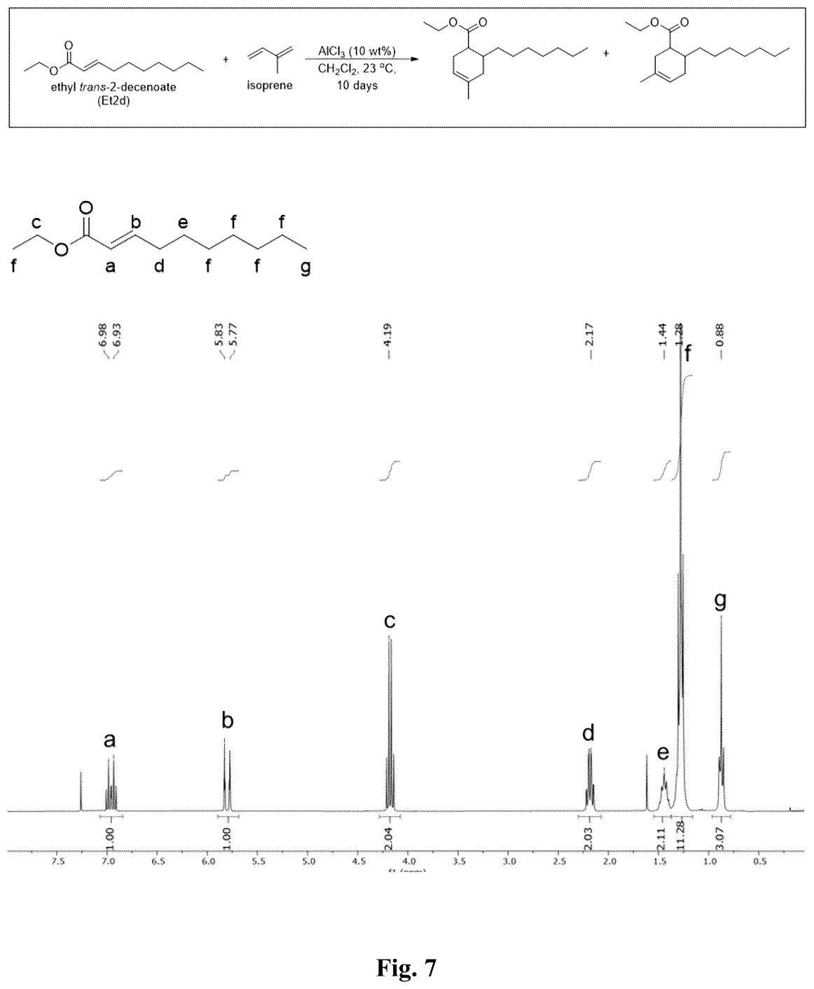

FIG. 8 provides NMR data for the reaction described in FIG. 7 after 8 days. The additional peaks with respect to FIG. 7 illustrate product formation.

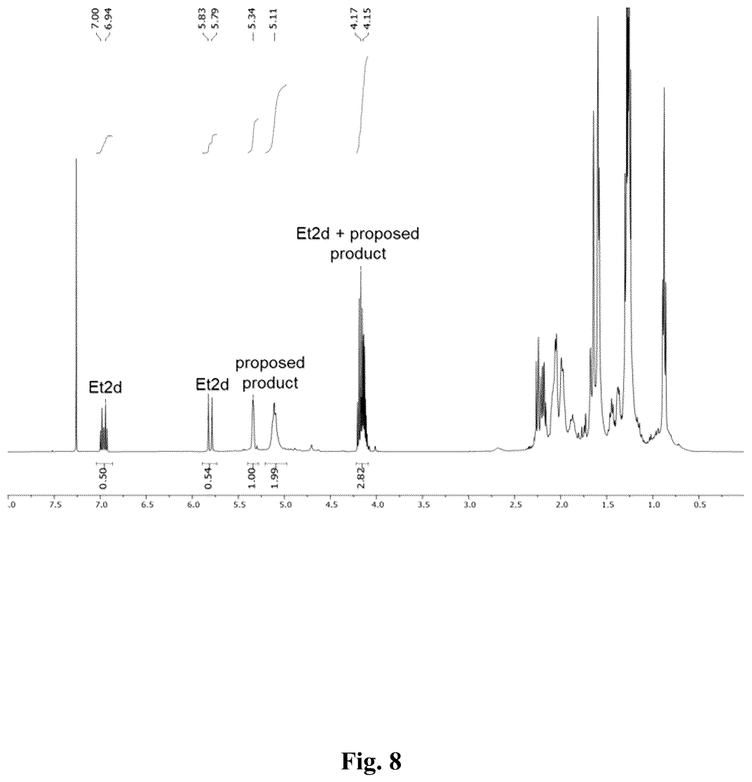

FIG. 9 provides NMR data for the reaction described in FIG. 7 an additional 2 days from the data in FIG. 8, after removing the reactant solvent. FIG. 9 illustrates that less than 25% of the Et2d reactant remains.

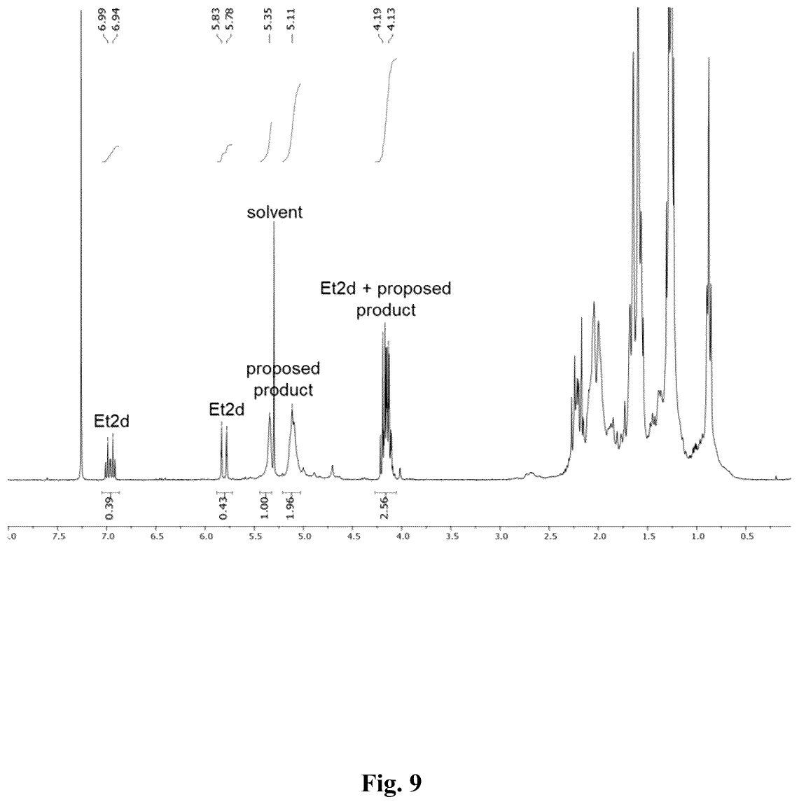

FIG. 10 provides NMR data for the reaction described in FIG. 7, where no olefin starting material remains.

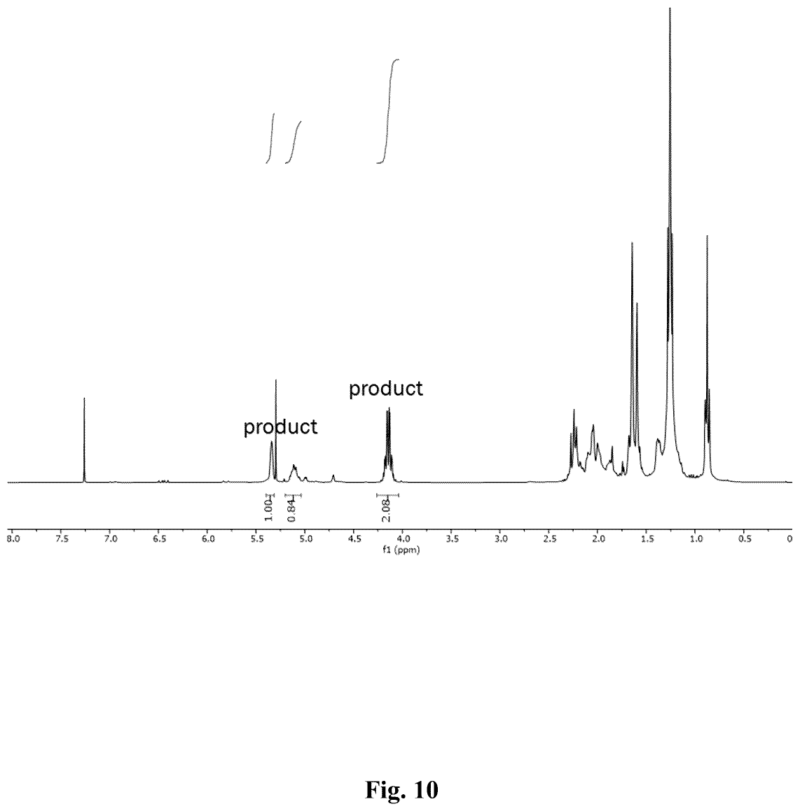

FIG. 11 provides gas chromatography-mass spectroscopy (GC-MS) data for the reaction described in FIG. 7.

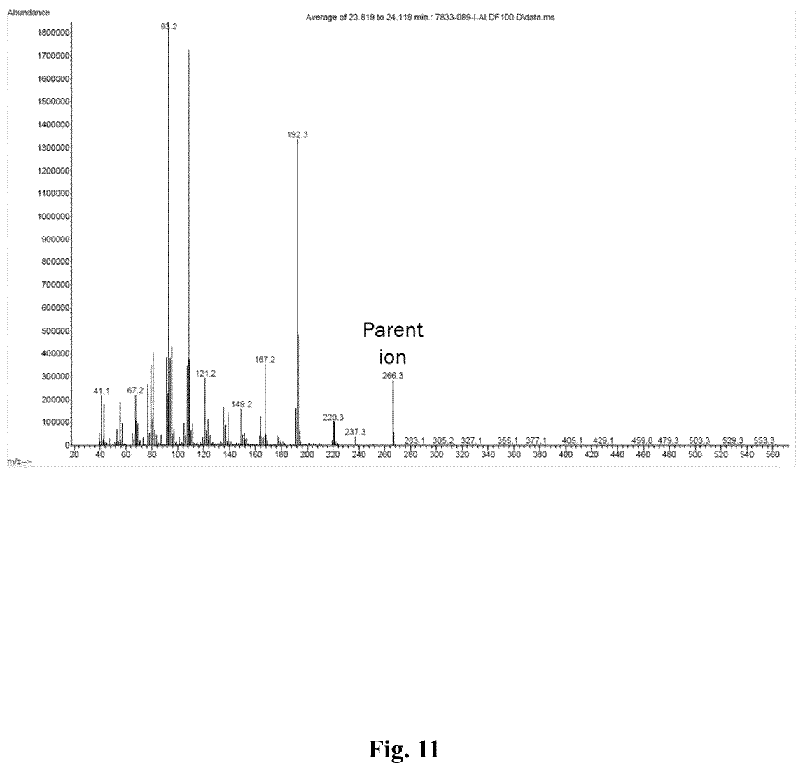

FIG. 12 provides an example fragmentation pattern for the reaction described in FIG. 7 for the GS-MS data in FIG. 11.

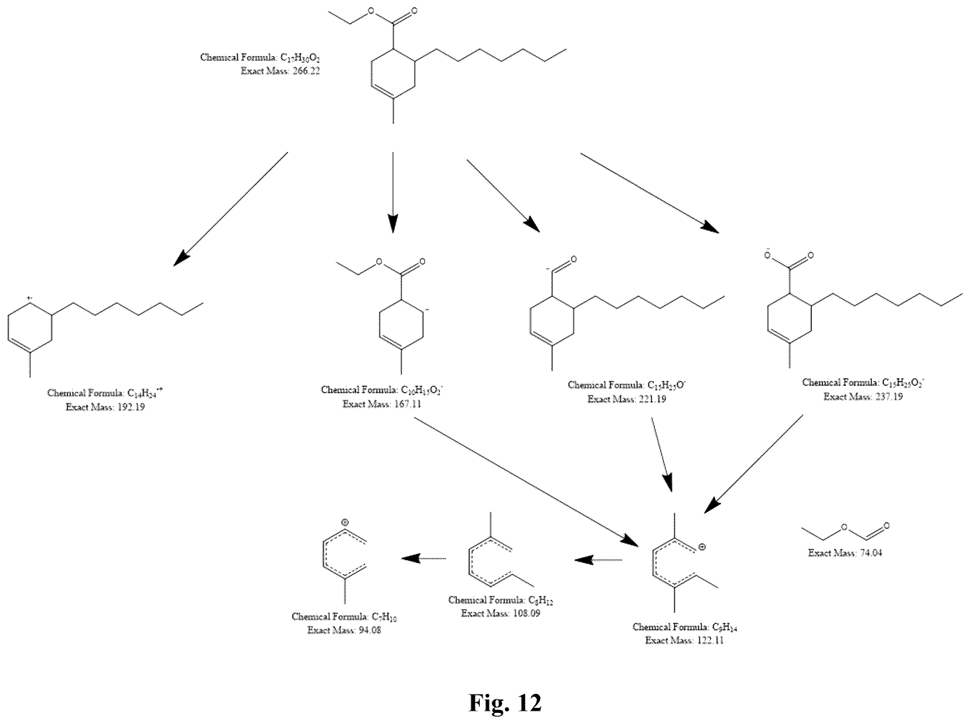

FIG. 13 provides an example reaction for generating cycloalkanes (top) and base NMR data after 7 days, indicating no starting material remains.

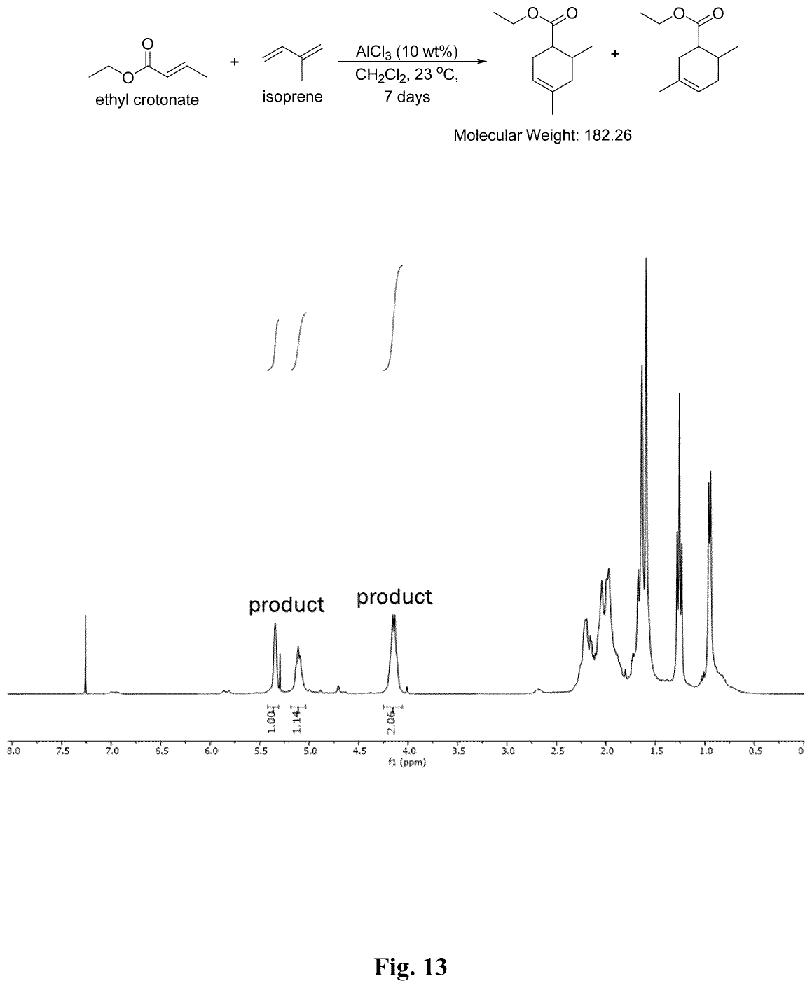

FIG. 14 provides GS-MS for the reaction provided in FIG. 13, with the marked peak showing the presence of the product parent ion.

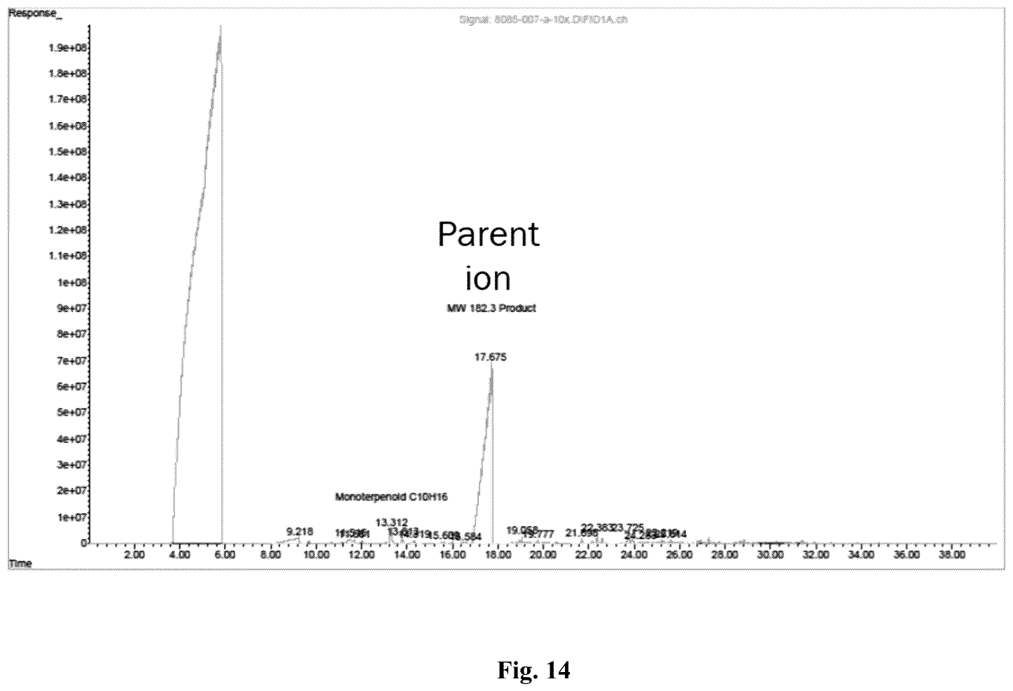

FIG. 15 provides an example scheme for the generation of cycloalkanes useful as sustainable aviation fuel (SAF).

FIG. 16 provides a product matrix illustrating products for the combination of various dienes and dienophiles, as described herein.

FIG. 17 provides NMR data for the provided reaction and illustrates a product purity greater than 95%.

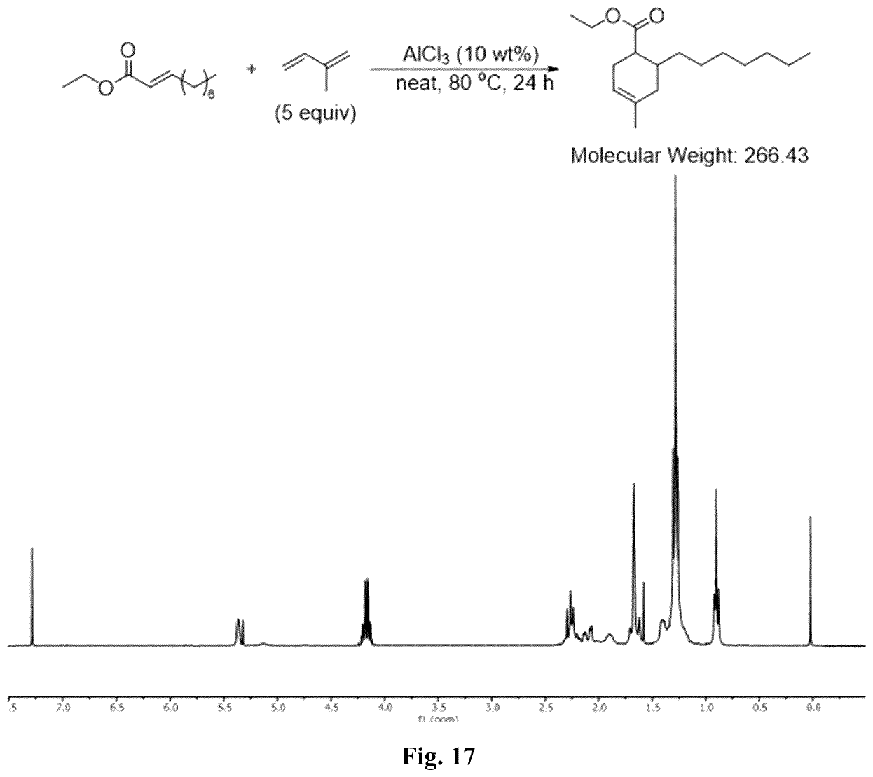

FIG. 18 provides GC-MS data for the reaction described in FIG. 17.

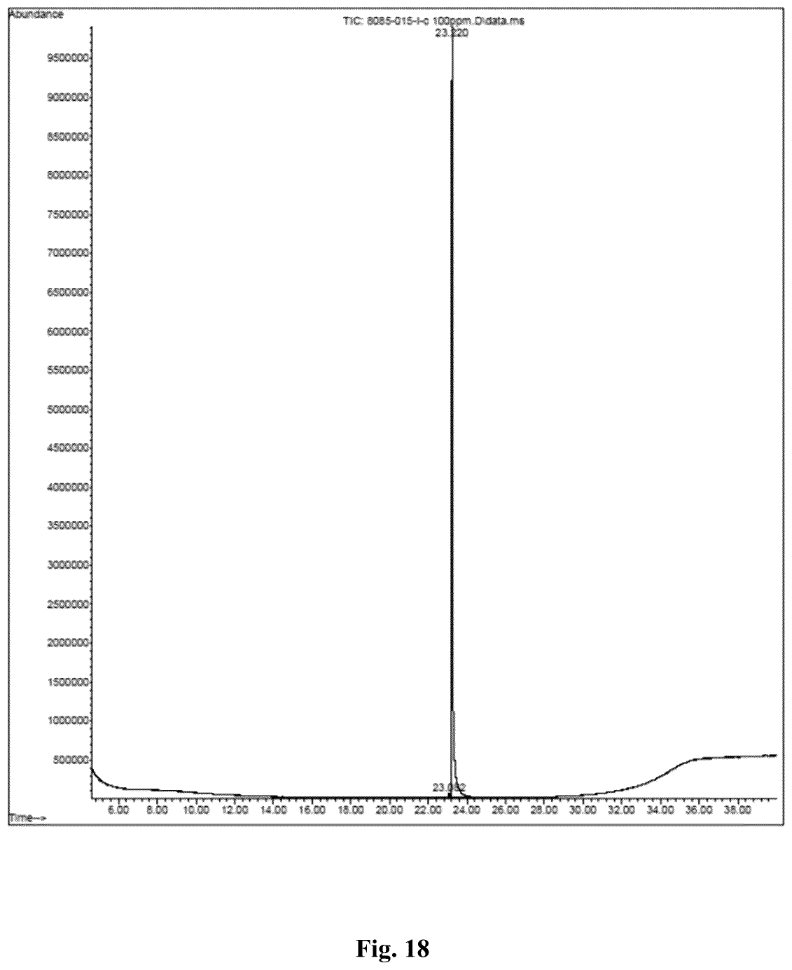

FIG. 19 provides NMR data for the provided reaction and illustrates a product purity greater than 80%.

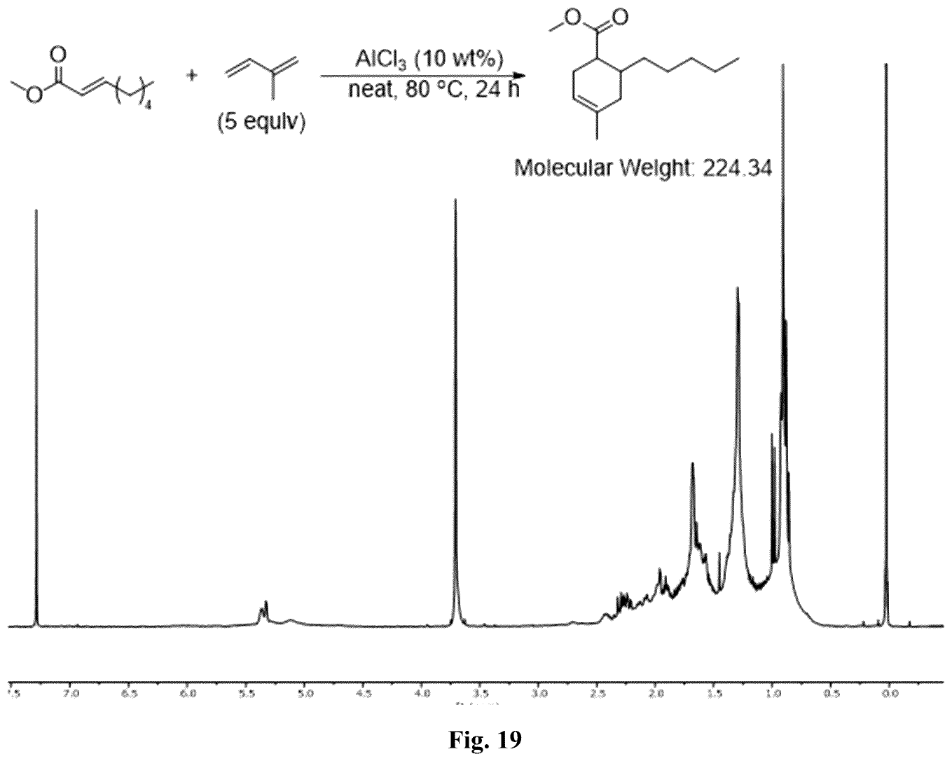

FIG. 20 provides NMR data for the provided reaction and illustrates a product purity greater than 80%.

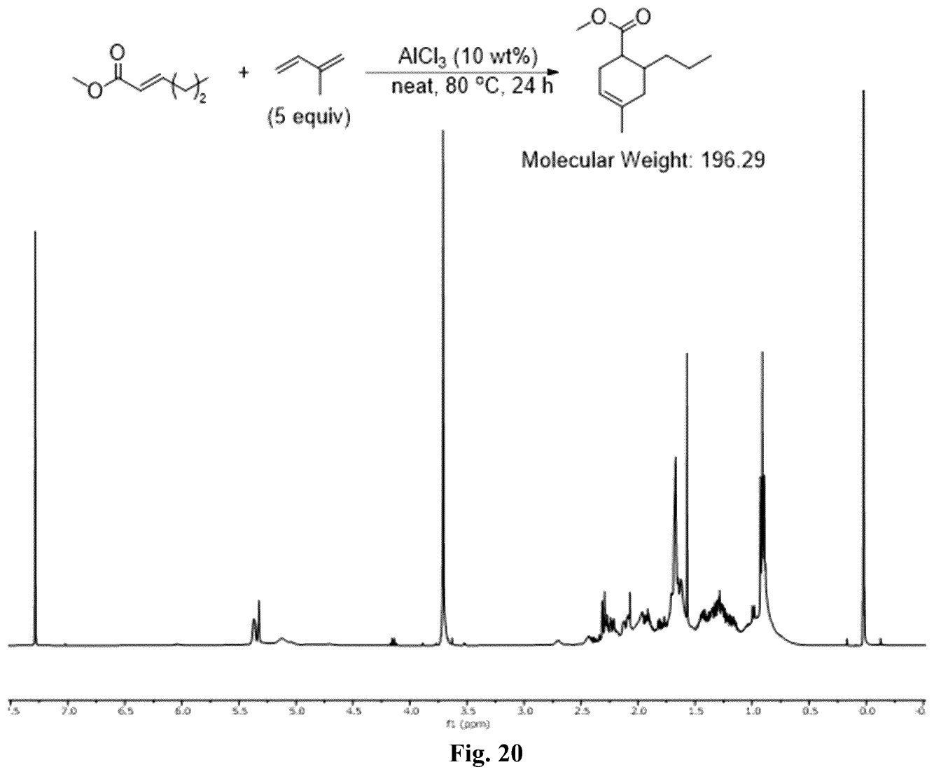

FIG. 21 provides NMR data for the provided reaction and illustrates a product purity greater than 90%.

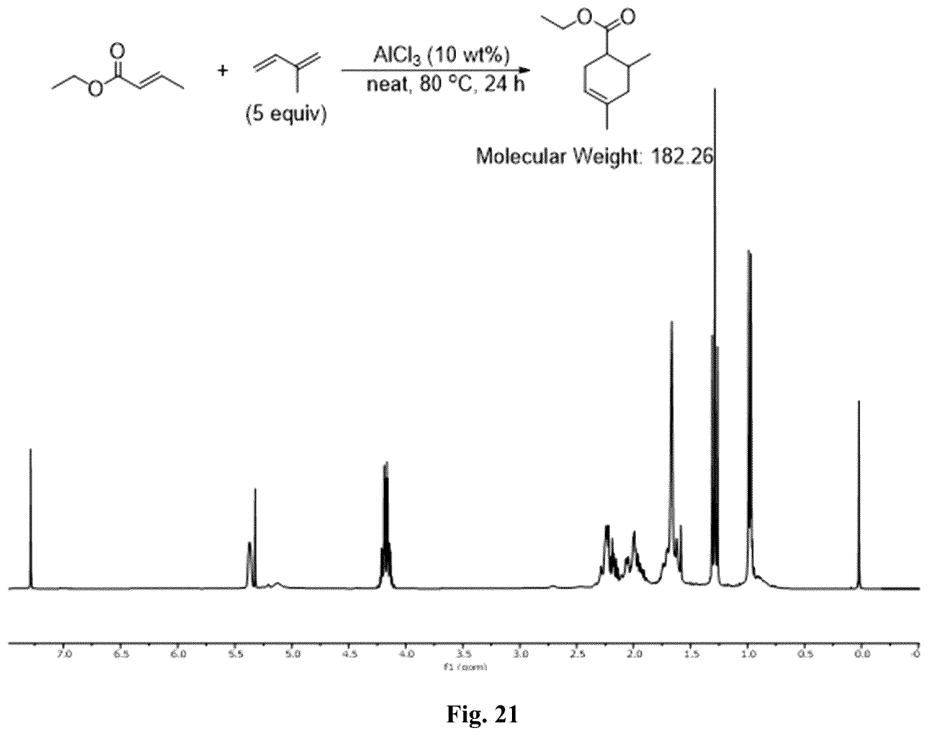

FIG. 22 provides GC-MS data for the reaction described in FIG. 21.

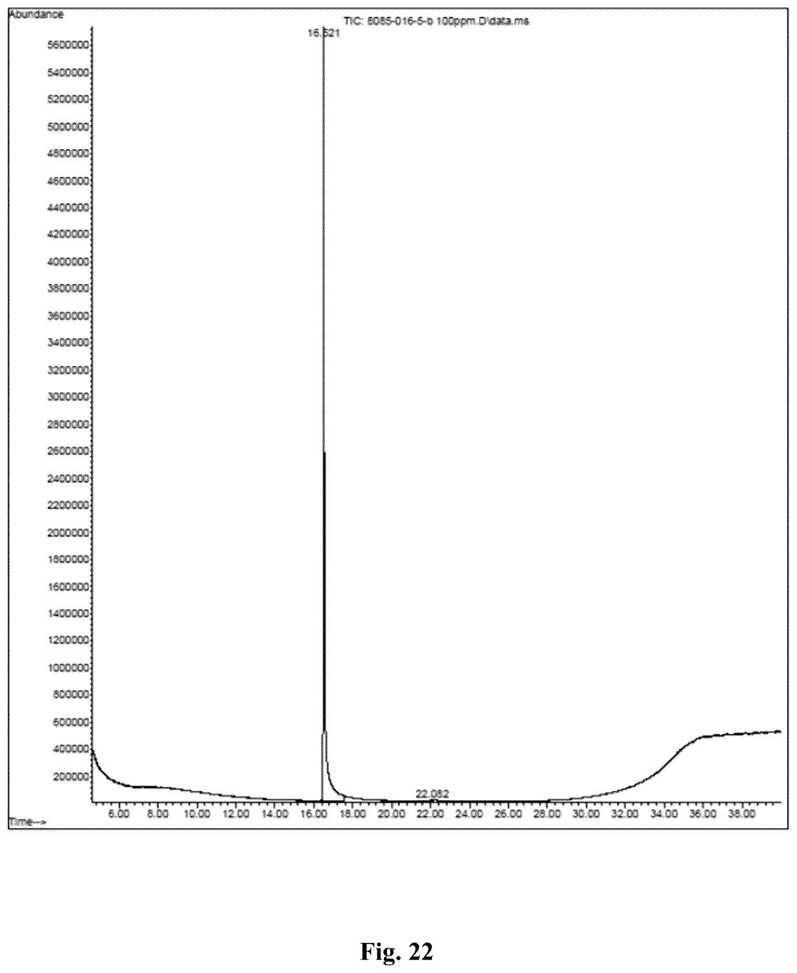

FIG. 23 provides NMR data for the provided reaction, illustrating partial hydrodeoxygenation which may be further optimized.

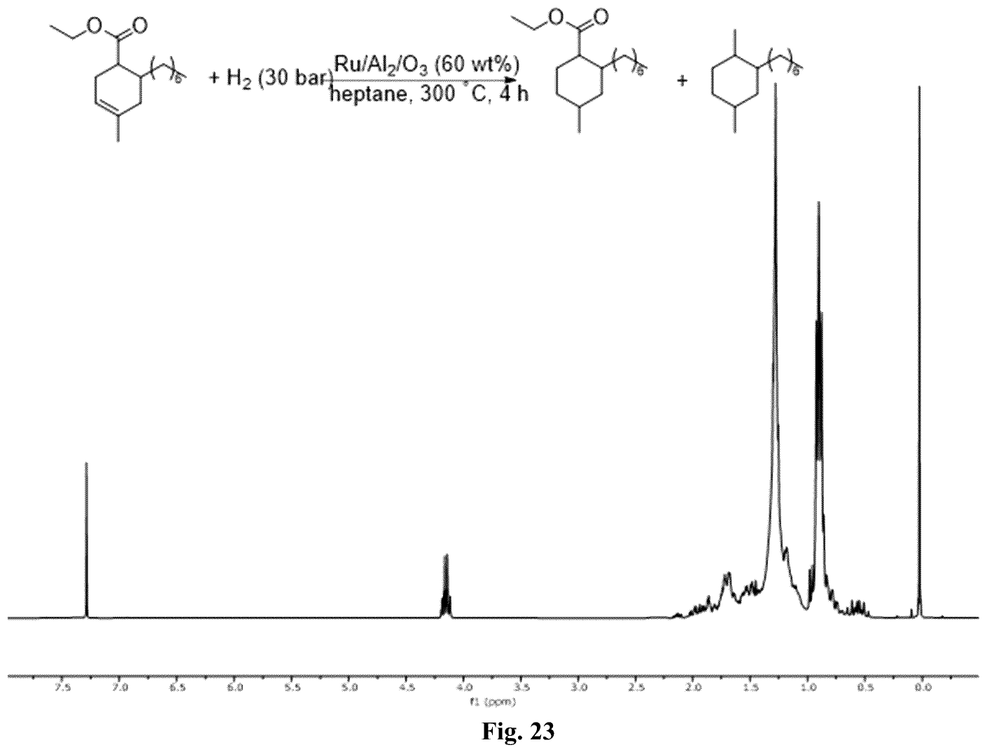

FIG. 24 provides NMR data for the provided reaction, illustrating partial hydrodeoxygenation which may be further optimized.

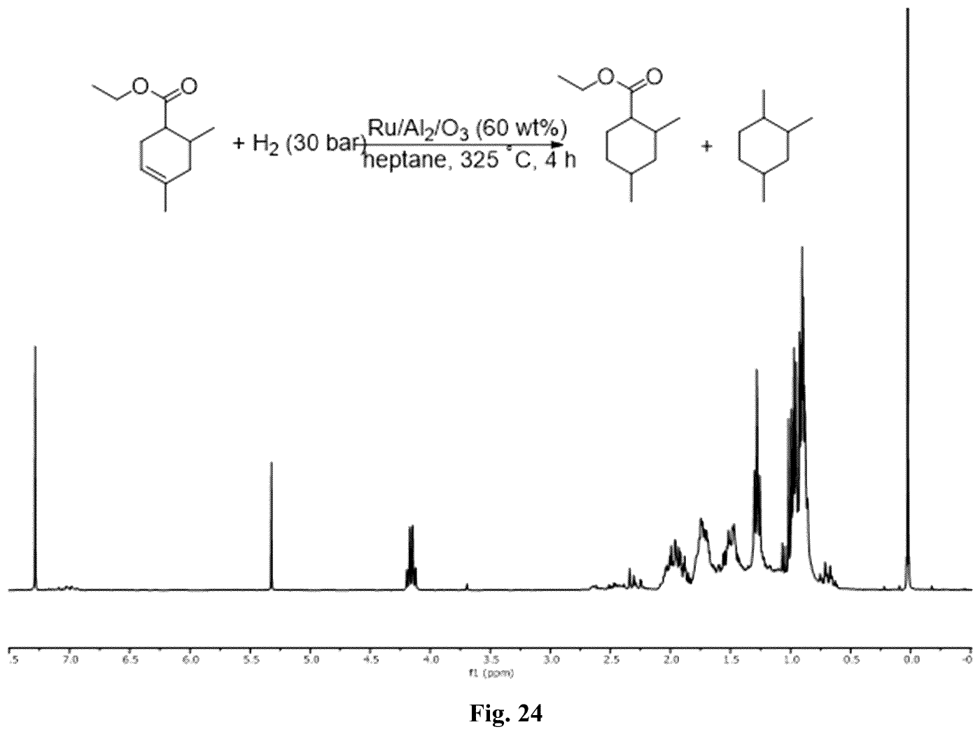

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

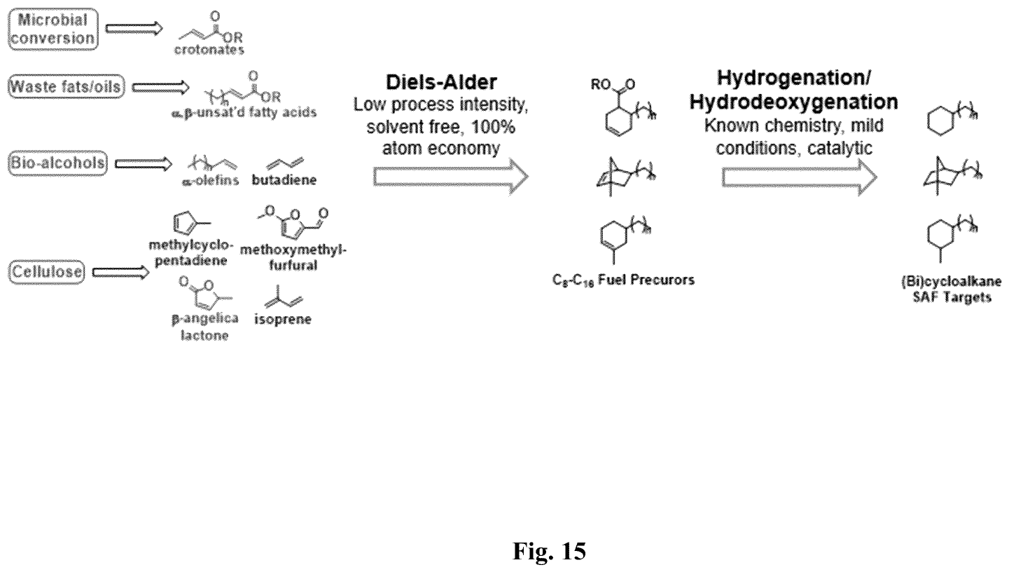

The present application is directed towards the generation of cycloalkanes useful as sustainable aviation fuel from various biological sources, including waste fats/oils, bio-alcohols, cellulose and products of microbial conversion. These waste sources can be used to make various dienes and dienophiles, which in turn can be reacted via an efficient Diels-Alder reaction to create fuel precursors. The fuel precursors are then subjected to hydrogenation or hydrodeoxygenation to remove excess O groups and form cycloalkanes. This process is outlined in FIG. 15.

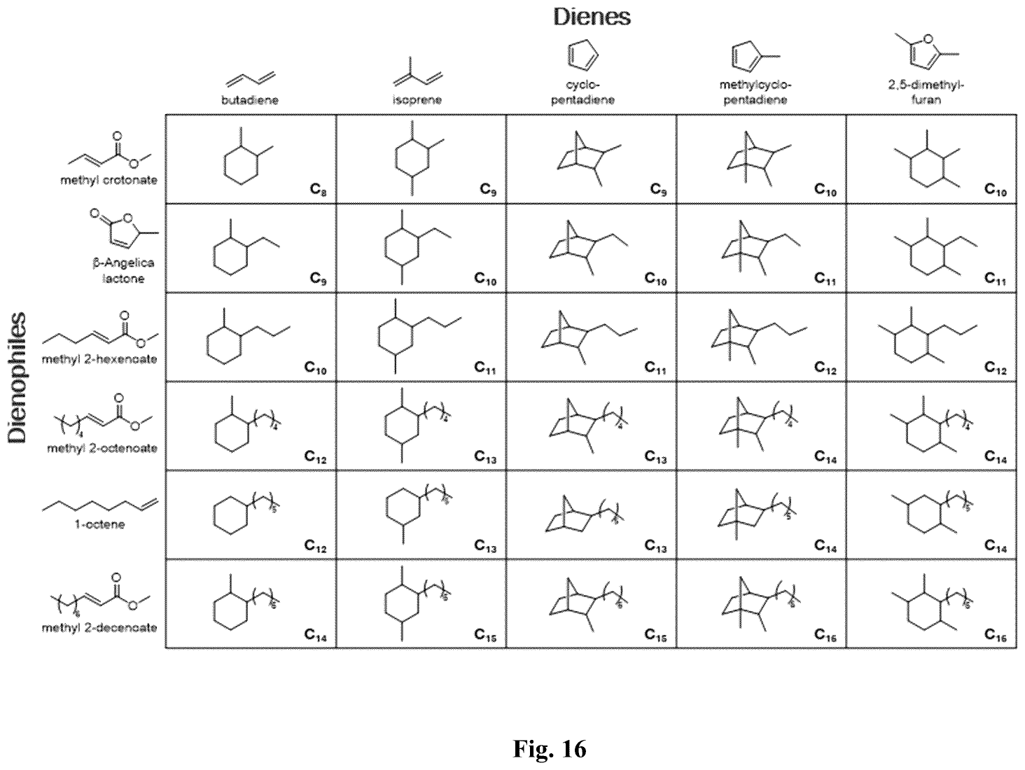

A description of the cycloalkane products along with the diene and dienophile reactants is illustrated in FIG. 16.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing lignocellulosic biomass;
converting a portion of the lignocellulosic biomass into one or more dienes comprising butadiene, isoprene, one or more cyclic dienes, or a combination thereof;
converting a portion of the lignocellulosic biomass into one or more dienophiles comprising olefins, unsaturated fatty acids, or a combination thereof;
reacting the one or more dienes and the one or more dienophiles in the presence of a catalyst, thereby generating one or more cyclic olefins; and
hydrogenating the one or more cyclic olefins, thereby generating one or more cyclic alkanes.

2. The method of claim 1, wherein the step of reacting the one or more dienes and one or more dienophiles is a catalyzed Diels-Alder reaction.

3. The method of claim 1, wherein the step of hydrogenating is performed via a catalyzed hydrogenation reaction.

4. The method of claim 1, wherein the step of converting a portion of lignocellulosic biomass into one or more dienes is performed in the presence of a Zn—Y catalyst.

5. The method of claim 1, wherein the step of converting a portion of lignocellulosic biomass into one or more dienophiles is performed in the presence of a Zn—Y catalyst and ethanol.

6. The method of claim 1, wherein the one or more cyclic alkanes are sustainable aviation fuel.

7. The method of claim 1, wherein the one or more dienes comprise butadiene.

8. The method of claim 1, wherein the one or more dienes comprise isoprene.

9. The method of claim 1, wherein the one or more dienes comprise one or more cyclic dienes defined by the formulas: 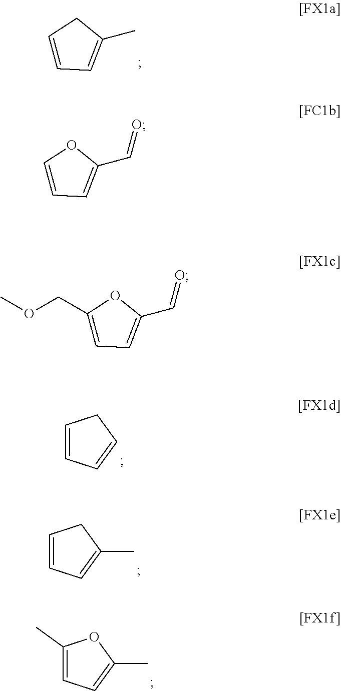
or a combination thereof.

10. The method of claim 1, wherein the one or more dienophiles comprise olefins defined by the formulas: 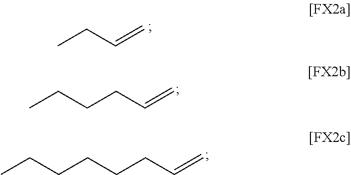
or a combination thereof.

11. The method of claim 1, wherein the one or more dienophiles comprise unsaturated fatty acids defined by the formulas: 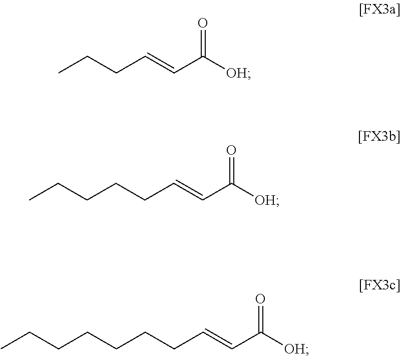
or a combination thereof.

12. The method of claim 1, wherein the one or more dienophiles comprise one or more molecules defined by the formulas: 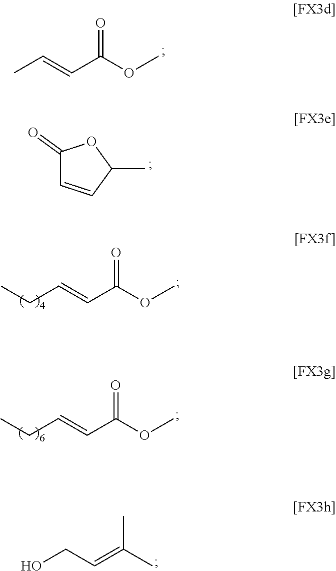
or a combination thereof.

13. The method of claim 1, wherein the one or more cyclic alkanes are defined by the formulas: 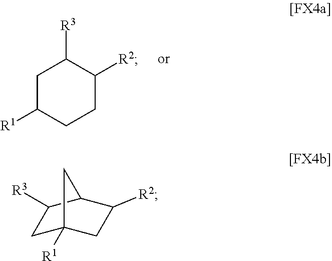
wherein each of R¹, R² and R³ are independently selected from an H atom or a C1-C8 alkane.

14. The method of claim 1, wherein the one or more cyclic alkanes are defined by the formulas: 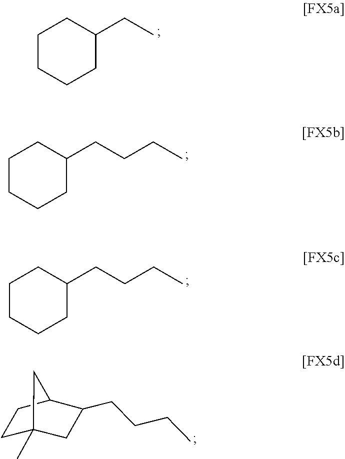
or a combination thereof.
